(19) United States
(12) Patent Application Publication
(10) Pub. No.: US 2004/0139972 A1
(43) Pub. Date: Jul. 22, 2004

(54) Stylet for use with endotracheal tubes having an articulable tip

(76) Inventors: Michael H. Wong, Irvine, CA (US); Jay S. Kotin, Irvine, CA (US)

Correspondence Address: LEONARD TACHNER, A PROFESSIONAL LAW, CORPORATION, 17961 SKY PARK CIRCLE, SUITE 38-E, IRVINE, CA, 92614

(21) Appl. No.: 10/749,018

(22) Filed: Dec. 29, 2003

Related U.S. Application Data
(63) Continuation-in-part of application No. 10/117,571, filed on Apr. 4, 2002.
Application No. 10/117,571 is a continuation-in-part of application No. 09/994,238, filed on Nov. 27, 2001, now Pat. No. 6,553,993.
Application No. 09/994,238 is a continuation-in-part of application No. 09/405,750, filed on Sep. 27, 1999, now Pat. No. 6,321,749.

(51) Int. Cl. A61M 16/00

(52) U.S. Cl. 128/207.14

(57) ABSTRACT

A fixed length, immalleable stylet for insertion into an endotracheal tube with an articulating tip. The stylet is preferably curved, semi-rigid and shape-retaining. This stylet is of fixed length and when inserted into an endotracheal tube with an articulating tip, promotes rigidity of the main body of the endotracheal tube, while allowing full function of the articulating tip mechanism. The stylet cannot protrude beyond the flexion point of an articulating tip endotracheal tube. This combination allows the tube to be passed in even very difficult airways while preserving the articulation capability afforded by the endotracheal tube.

### BACKGROUND OF THE INVENTION

1. Cross-Ref r nce to Related Applications

This application is a continuation-in-part of U.S. application Ser. No. 10/117,571 filed on Apr. 4, 2002 (now U.S. Pat. No. ______) which is a continuation-in-part of U.S. application Ser. No. 09/994,238 filed on Nov. 26, 2001 (now U.S. Pat. No. 6,553,993), which is a continuation-in-part of U.S. application Ser. No. 09/405,750 filed on Sep. 27, 1999 (Now U.S. Pat. No. 6,321,749).

2. Field of the Invention

The invention generally relates to stylets inserted into endotracheal tubes to promote tube rigidity and manipulation of the tip of the tube. Conventional stylets are malleable and have a length that allows passage through the entire length of an endotracheal tube. The stylet of the present invention is shorter than the endotracheal tube into which it is designed to be inserted. It is of a pre-set, non-adjustable length and is for use with an endotracheal tube that has an articulating tip.

### BACKGROUND ART

A stylet is typically inserted into the lumen of an endotracheal tube if it is determined that a patient has a difficult airway. Current stylets are usually malleable. (See U.S. Pat. No. 3,996,939 to Sheridan and Jackson). This malleability allows an airway practitioner to bend the tip of stylet and thus the endotracheal tube into a hockey stick or J-shape position to facilitate passage of the tube tip into the airway of a patient. The endotracheal tube with a flexion mechanism allows this tip manipulation to be performed without the traditional stylet. This is favorable since stylets are associated with airway injuries, difficulty of removal, increased critical time requirements and increased cost of the procedure. With the most difficult airways, there is a need for an absolute rigid tube body. The current state of the art consists of malleable stylets, usually comprising a malleable metal that can be placed in the lumen of an endotracheal tube and then must be bent carefully without protrusion through the distal end of the endotracheal tube. Protrusion through the distal end creates potential for serious injury to the airway. Bending the stylet into the “J” shape creates difficulty for removal during a very critical time in the intubation process.

Improved endotracheal tubes with features that allow flexion at the distal end to aid in the intubation process are disclosed in U.S. Pat. Nos. 6,321,749 and 6,553,993 to Toti et al. With the advent of an endotracheal tube with an articulating tip, the traditional stylet is no longer always needed. This tube creates the required tip manipulation without the use of the traditional stylet. In the most difficult airways however, there exists a need for a device to provide absolute rigidity of the body of the endotracheal tube. There have been stylets introduced to aid in the manipulation of the more conventional endotracheal tube tip. (See U.S. Pat. No. 4,329,983 to Fletcher and No. 5,259,377 to Schroeder). Flexion is not needed in the stylet of the present invention since the flexion is inherent in the tip flexing endotracheal tube with which the invention herein is specifically designed to be used.

### SUMMARY OF THE INVENTION

The stylet of the present invention consists of an immalleable, preferably curved rod, which is inserted into the proximal end of an endotracheal tube with an articulating tip. The stylet may have a curved shape that matches the inherent curve (commonly called a Magill curve) of the endotracheal tube. There is also a version that is straight. There exists an enlarged flange/stop on the proximal end of the stylet that allows insertion into the tube only until the distal tip of the stylet reaches the flexion point of the articulating tip endotracheal tube. This proximal flange also facilitates rapid removal from the endotracheal tube. There are current stylets that employ mechanisms to limit the depth that they can be inserted. (See U.S. Pat. No. 4,185,639 to Linder and No. 3,957,055 to Zimmerman and Linder). The stylet of the present invention, by virtue of its preformed curvature, does not create any friction within the tube lumen; consequently, removal is rapid and simple. Since the distal tip of the fixed length of the invention ends before the flexion point of the articulating tip endotracheal tube, the tube can be fully manipulated and absolute tube rigidity is achieved. At the distal tip of the invention there is a small flange that when inserted into the endotracheal tube rests at the back portion of the tube, opposite the articulation point. When the distal tip is articulated, this flange assists in maintaining the rigidity of the tube, and limits any “play” or movement of the stylet within the lumen. This proximal flange also facilitates rapid removal from the endotracheal tube. The stylet is preferably constructed as a single injection molded rod that is designed to fit into the lumen of the endotracheal tube and terminates just proximal to the articulation point. The stylet of the present invention, by virtue of its preformed curvature and self-lubricating biocompatible polymers used in its construction, can be inserted and removed from the lumen of the endotracheal tube rapidly and with a minimum amount of friction. The straight version of the tube has less friction than standard coated and non-coated rigid and malleable stylets because of the nature of the self-lubricating biocompatible polymers used in its construction and the design of its distal tip. Since the distal tip of the invention ends before the flexion point of the endotracheal tube, the tube can be fully manipulated and maximum tube rigidity can be achieved. The distal tip ends in a rounded shape and the tip angles downward, thus imparting slight pressure against the posterior wall of the endotracheal tube. In accordance with one specific, exemplary embodiment of the invention, the advantageous features include:

1. Pre-curved stylet made to match an articulating tip endotracheal tube curvature;

2. Use of a polymer, co-polymer, composite, or other similar semi-rigid immalleable material;

3. Proximal end flange creating limit of insertion length;

4. Proximal end ring or flange to facilitate rapid removal;

5. An enlarged distal tip that presses back against the back wall of the flexion point of the endotracheal tube;

6. Insertion length limited so that the distal tip cannot pass the flexion point of an articulating tip endotracheal tube and thus cannot extend beyond the distal end of the tube where otherwise there could be injury to the airway during insertion of the tube.

### BRIEF DESCRIPTION OF THE DRAWINGS

The aforementioned objects and advantages of the present invention, as well as additional objects and advantages thereof, will be more fully understood hereinafter as a result of a detailed description of a preferred embodiment when taken in conjunction with the following drawings in which:

FIG. 1 is an elevational view of the combined endotracheal tube and stylet of the present invention;

FIG. 2 is an enlarged view of a preferred embodiment of the inventive stylet;

FIG. 3 is a view of a preferred embodiment of a stop flange and removing handle of an inventive stylet;

FIG. 4 is an end view of the combination of FIG. 1;

FIG. 5 is a view of a first alternative embodiment of a removing handle;

FIG. 6 is a view of a second alternative embodiment of a removing handle;

FIG. 7 is an elevational view of a second embodiment of an inventive stylet; and

FIG. 8 is a view of the second embodiment stylet, but shown with an alternative combined stop flange and removing handle.

### DETAILED DESCRIPTION OF PREFERRED EMBODIMENTS

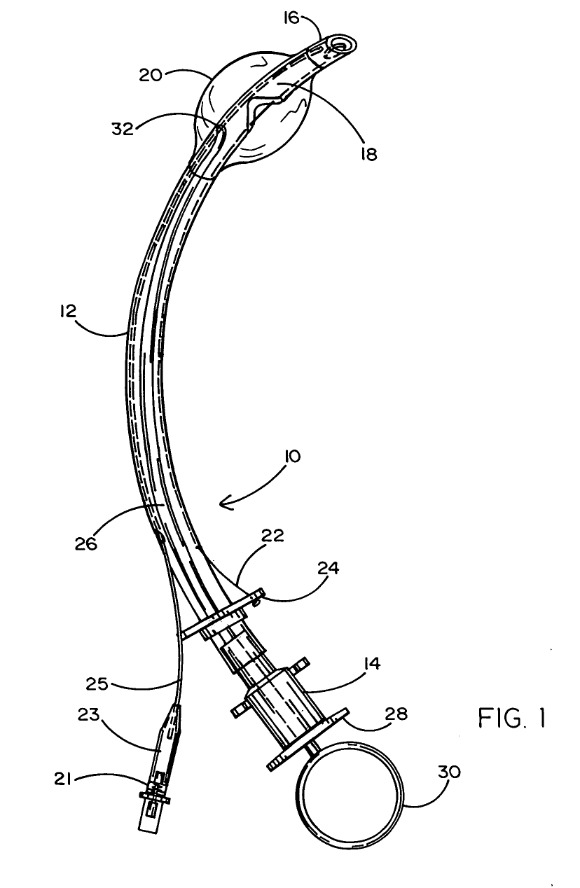

Referring to the accompanying figures and to FIG. 1 in particular, it will be seen that a combined endotracheal tube and stylet 10 comprises a tracheal tube 12 having a proximal end and a distal end 16. Tube 12 has an articulable tip 18 and an inflatable cuff 20, the latter functioning in conjunction with valve 21, pilot balloon 23 and thin tube 25 as described in U.S. Pat. No. 6,321,749 the content of which is hereby incorporated herein by reference as if fully set forth herein. Tip 18 is controlled by a control mechanism 24 closer to the proximal end 14 and acting through a cable 22 which is routed to the distal end 16 inside the outer wall of tube 12 to remotely articulate the tip for entry into a patient's airway.

To further ease the entry of the tube 12 into difficult airways, one may insert stylet 26 into the interior of the tube as shown in FIG. 1. Stylet 26 rigidizes the tube so that force can be applied to the tube as the distal end is fed into the airway. Unlike conventional stylets, stylet 26 is uniquely configured to be used with the endotracheal tube having an articulable tip. By limiting its length and employing a stop flange 28 which contacts the proximal end 14, stylet 26 can extend in tube 12 only until stylet tip 32 can reach but not go beyond articulation tip 18. Tip 32 is preferably enlarged, flattened and/or bent to engage the posterior inner tube wall surface proximate the articulable tip 18. Stylet tip 32 while in contact with the interior tube wall, facilitates articulation of tip 18 thus enhancing operation of tracheal tube 12 during manipulation into an airway.

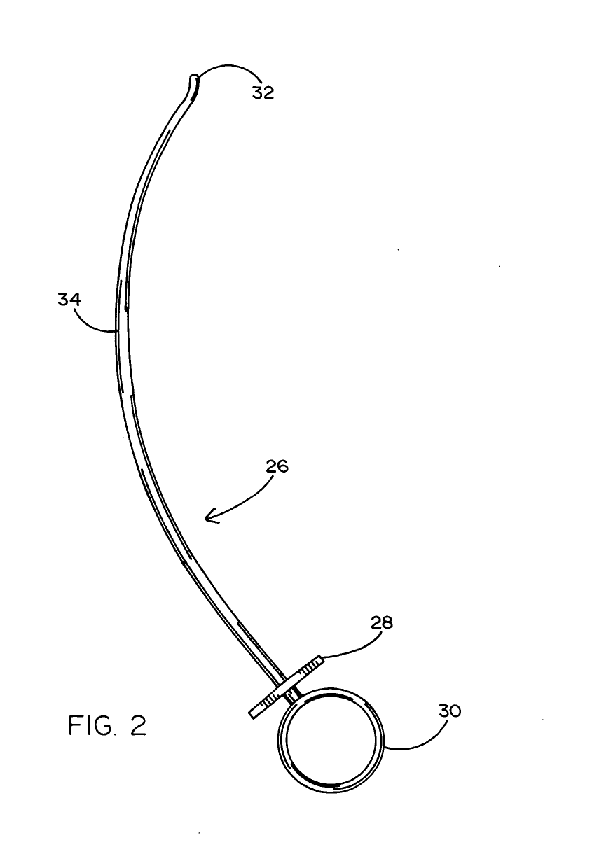

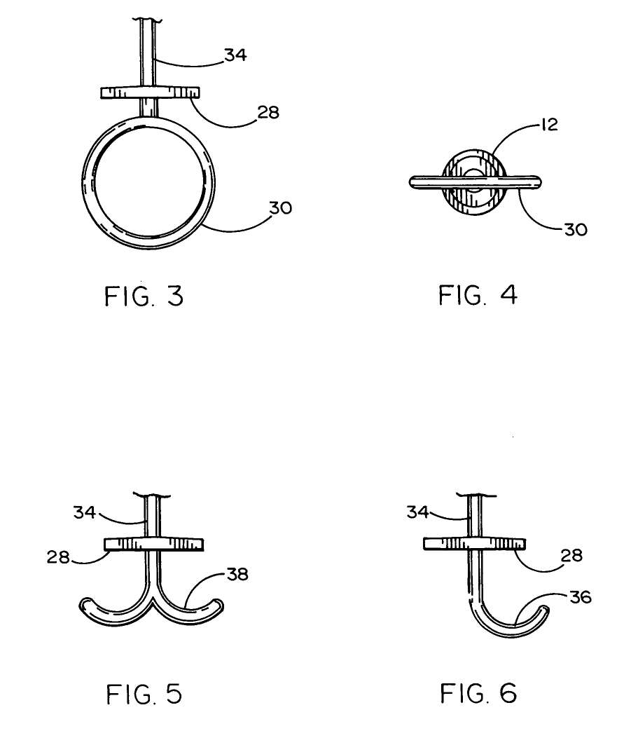

Stylet 26 is preferably of a diameter in the range of 2 to 11 millimeters and is curved to have the same shape as tracheal tube 12 to facilitate smooth, low-friction entry and removal. Because the stylet is made of an immaleable polymer rod that is inherently semi-rigid and shape retaining, it tends to hold this curvature throughout manipulation of the tube and stylet during airway entry. Withdrawal of the stylet is further facilitated by a removing handle 30 seen best in FIGS. 2-4 which further illustrate the embodiment of FIG. 1. Alternative J-shaped removing handles 36 in FIGS. 6 and 38 in FIG. 5 are also contemplated. Clearly, any handle for grasping by at least one finger would be suitable for use in aiding the withdrawal of the stylet from the tube after intubation.

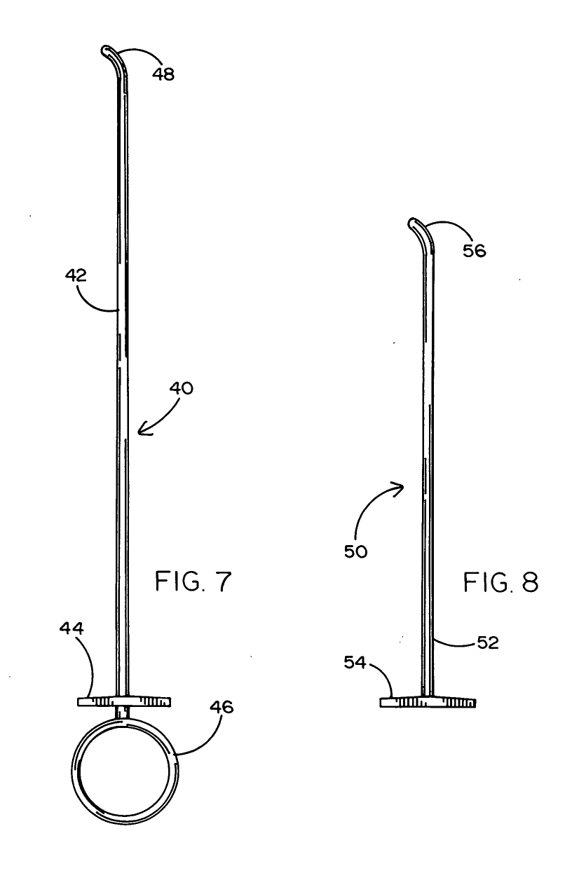

As seen in FIGS. 7 and 8, the stylet of the present invention may also comprise a straight rod. As shown in FIG. 7, a stylet 40 comprises a straight rod 42 terminating at a proximate end in a stop flange 44 and withdrawing handle 46 and terminating at a distal end in a bent tip 48. In FIG. 8, a stylet 50 comprises a straight rod 52 terminating at a proximate end in a combined stop flange and withdrawing handle 54 end terminating at a distal end in an enlarged and bent tip 56.

It will now be apparent that the present invention comprises a uniquely configured stylet for use with an endotracheal tube having an articulating tip. Although preferred embodiments of the invention have been disclosed herein, various modifications and additions will be perceived by those having the benefit of the teaching herein. Accordingly, it will be understood that the scope hereof is to be limited only by the appended claims and their equivalents.

We claim:

1. A stylet for use with an endotracheal tube having an articulating tip; the stylet comprising:
an elongated immaleable rod having a diameter that is less than the inner diameter of said tube and having a length that is shorter than said tube.

2. The stylet recited in claim 1 wherein said rod is shaped to conform to the curvature of said tube.

3. The stylet recited in claim 1 further comprising a limiting device affixed to the proximal end of said rod for engaging the proximal end of said tube to limit the insertion of said rod into said tube.

4. The stylet recited in claim 3 wherein said limiting device is shaped for grasping by at least one finger of a human hand.

5. The stylet recited in claim 1 wherein said rod has a distal end having an enlargement.

6. The stylet recited in claim 1 wherein said rod has a distal end which is flattened.

7. The stylet recited in claim 1 wherein said rod has a bent distal end tip.

8. The stylet recited in claim 1 wherein said rod has a uniform straight shape over most of its length.

9. The stylet recited in claim 1 wherein said rod has a diameter which is in the range of 2 mm to 11 mm.

10. The stylet recited in claim 1 wherein said rod is made of biocompatible polymer.

11. In combination with an endotracheal tube having an articulable distal portion for manipulation during insertion into an airway, a stylet for insertion into the tube to rigidize the tube along the tube length not including the articulable distal portion; the stylet comprising:
an elongated, shape-retaining, semi-rigid rod having a diameter smaller than the inner diameter of said tube and having a length that is shorter than said tube.

12. The stylet recited in claim 11 wherein said rod is shaped to conform to the curvature of said tube.

13. The stylet recited in claim 11 further comprising a limiting device affixed to the proximal end of said rod for engaging the proximal end of said tube to limit the insertion of said rod into said tube.

14. The stylet recited in claim 13 wherein said limiting device is shaped for grasping by at least one finger of a human hand.

15. The stylet recited in claim 11 wherein said rod has a distal end having an enlargement.

16. The stylet recited in claim 11 wherein said rod has a distal end which is flattened.

17. The stylet recited in claim 11 wherein said rod has a bent distal end tip.

18. The stylet recited in claim 11 wherein said rod has a uniform straight shape over most of its length.

19. The stylet recited in claim 11 wherein said rod has a diameter which is in the range of 2 mm to 11 mm.

20. The stylet recited in claim 11 wherein said rod is made of biocompatible polymer.

21. The stylet recited in claim 11 wherein the length of said rod is selected to permit the distal end of said stylet to be proximate said articulable distal portion of said tube when the proximal end of said rod is substantially aligned with the proximal end of said tube.
